SEQUENCE LISTING (24 sequences)

SEQ ID NO: 1 - RNA, 10 nt, synthetic construct
  misc_feature 1..10: note = Apt-1
  source 1..10: mol_type = other RNA
cggtgttgct

SEQ ID NO: 2 - RNA, 10 nt, synthetic construct
  misc_feature 1..10: note = Apt-2
  source 1..10: mol_type = other RNA
gtggtccccg

SEQ ID NO: 3 - RNA, 10 nt, synthetic construct
  misc_feature 1..10: note = Apt-3
  source 1..10: mol_type = other RNA
cgctgtggtc

SEQ ID NO: 4 - RNA, 10 nt, synthetic construct
  misc_feature 1..10: note = Apt-4
  source 1..10: mol_type = other RNA
agctgtggcc

SEQ ID NO: 5 - RNA, 10 nt, synthetic construct
  misc_feature 1..10: note = Apt-5
  source 1..10: mol_type = other RNA
cgctggtgct

SEQ ID NO: 6 - RNA, 10 nt, synthetic construct
  misc_feature 1..10: note = Apt-6
  source 1..10: mol_type = other RNA
cgctgtggct

SEQ ID NO: 7 - RNA, 10 nt, synthetic construct
  misc_feature 1..10: note = Apt-7
  source 1..10: mol_type = other RNA
cggcgttgtt

SEQ ID NO: 8 - RNA, 10 nt, synthetic construct
  misc_feature 1..10: note = Apt-8
  source 1..10: mol_type = other RNA
cggtgtaggt

SEQ ID NO: 9 - RNA, 10 nt, synthetic construct
  misc_feature 1..10: note = Apt-9
  source 1..10: mol_type = other RNA
ctctgtggtg

SEQ ID NO: 10 - RNA, 10 nt, synthetic construct
  misc_feature 1..10: note = Apt-10
  source 1..10: mol_type = other RNA
gtggtcgctg

SEQ ID NO: 11 - RNA, 10 nt, synthetic construct
  misc_feature 1..10: note = Apt-11
  source 1..10: mol_type = other RNA
ggggtggggc

SEQ ID NO: 12 - RNA, 10 nt, synthetic construct
  misc_feature 1..10: note = Apt-12
  source 1..10: mol_type = other RNA
cgaggccggg

SEQ ID NO: 13 - RNA, 10 nt, synthetic construct
  misc_feature 1..10: note = Apt-13
  source 1..10: mol_type = other RNA
gcggggcccg

SEQ ID NO: 14 - DNA, 10 bp, synthetic construct
  misc_feature 1..10: note = Apt-14
  source 1..10: mol_type = other DNA
cggtgttgct

SEQ ID NO: 15 - DNA, 10 bp, synthetic construct
  misc_feature 1..10: note = Apt-15
  source 1..10: mol_type = other DNA
gtggtccccg

SEQ ID NO: 16 - DNA, 10 bp, synthetic construct
  misc_feature 1..10: note = Apt-16
  source 1..10: mol_type = other DNA
cgctgtggtc

SEQ ID NO: 17 - DNA, 10 bp, synthetic construct
  misc_feature 1..10: note = Apt-17
  source 1..10: mol_type = other DNA
agctgtggcc

SEQ ID NO: 18 - DNA, 10 bp, synthetic construct
  misc_feature 1..10: note = Apt-18
  source 1..10: mol_type = other DNA
cgctggtgct

SEQ ID NO: 19 - DNA, 10 bp, synthetic construct
  misc_feature 1..10: note = Apt-19
  source 1..10: mol_type = other DNA
cgctgtggct

SEQ ID NO: 20 - DNA, 10 bp, synthetic construct
  misc_feature 1..10: note = Apt-20
  source 1..10: mol_type = other DNA
cggcgttgtt

SEQ ID NO: 21 - DNA, 10 bp, synthetic construct
  misc_feature 1..10: note = Apt-21
  source 1..10: mol_type = other DNA
cggtgtaggt

SEQ ID NO: 22 - DNA, 10 bp, synthetic construct
  misc_feature 1..10: note = Apt-22
  source 1..10: mol_type = other DNA
ctctgtggtg

SEQ ID NO: 23 - DNA, 10 bp, synthetic construct
  misc_feature 1..10: note = Apt-23
  source 1..10: mol_type = other DNA
gtggtcgctg

SEQ ID NO: 24 - RNA, 25 nt, synthetic construct
  source 1..25: mol_type = other RNA
gctggggtggggcggatcggtgttg

(19) United States
(12) Patent Application Publication
(10) Pub. No.: US 2025/0327081 A1
(43) Pub. Date: Oct. 23, 2025

(54) TDP-43-BINDING SINGLE-STRANDED APTAMERS AND USES THEREOF

(71) Applicants: FONDAZIONE ISTITUTO ITALIANO DI TECNOLOGIA, GENOVA (IT); FUNDACIÓ CENTRE DE REGULACIÓ GENÒMICA, BARCELONA (ES); INSTITUCIÓ CATALANA DE RECERCA I ESTUDIS AVANÇATS, BARCELONA (ES); KING'S COLLEGE LONDON, LONDON (GB); THE UNIVERSITY COURT OF THE UNIVERSITY OF EDINBURGH, EDINBURGH (GB)

(72) Inventors: Gian Gaetano TARTAGLIA, GENOVA (IT); Alexandros ARMAOS, BARCELONA (ES); Fernando CID, BARCELONA (ES); Natalia SANCHEZ DE GROOT, SABADELL, BARCELLONA (ES); Ricardo GRANA-MONTES, PASCAIS, Ourense (ES); Annalisa PASTORE, LONDON (GB); Elsa ZACCO, GENOVA (IT); Mathew HORROCKS, EDINBURGH (GB); Owen KANTELBERG, STEVENAGE (GB)

(21) Appl. No.: 18/864,219

(22) PCT Filed: May 9, 2023
(86) PCT No.: PCT/EP2023/062203

§ 371 Date: Nov. 8, 2024

(30) Foreign Application Priority Data
May 9, 2022 (IT) 102022000009500

(51) Int. Cl. C12N 15/115; A61P 25/28; G01N 33/68

(52) U.S. Cl. CPC C12N 15/115; A61P 25/28; G01N 33/6896; C12N 2310/16; G01N 2333/4703; G01N 2800/2821

(57) ABSTRACT

The invention relates to a short single-stranded DNA or RNA aptamer that is capable of binding the TDP-43 protein and of detecting all of the different TDP-43 structures individually, from the soluble monomer to the TDP-43 larger aggregates. The aptamer of the invention is also capable of inhibiting aggregation of TDP-43. Because of these properties, the RNA aptamer of the invention is suitable for use in both the diagnosis and therapeutic treatment and prevention of TDP-43-related proteinopathies, such as ALS and FTD.

### FIELD OF THE INVENTION

The present invention relates to isolated single-stranded aptamers suitable for use in the diagnosis and therapeutic treatment of neurodegenerative diseases such as amyotrophic lateral sclerosis (ALS) and frontotemporal dementia (FTD), and as detection probes for investigating the molecular mechanisms underlying the aforementioned diseases.

### BACKGROUND OF THE INVENTION

In neurodegenerative diseases such as ALS and FTD, the TAR DNA-binding protein 43 (also known as “TDP-43”), which is normally localized to the cell nucleus, may mislocalize to the cytoplasm of affected neurons and glia and, over time, form toxic aggregates that gradually increase in size (Ratti and Buratti, 2016). The lack of specific and sensitive detection means capable of identifying the pool of different TDP-43 aggregates up to now has limited the comprehension of the key steps of the TDP-43 aggregation process. For this reason, historically, many trials have failed in tracking the progression of TDP-43-associated neurodegenerative diseases, in particular ALS. Current neuropathological assessments solely rely on the semi-quantitative evaluation of mature TDP-43 cytoplasmic aggregates, which however are not reliable pathological indicators of neurotoxicity (Gregory et al., 2020). The characterization of individual TDP-43 aggregates would be more informative than looking at the micrometer-large TDP-43 cytoplasmic aggregates, as is currently performed.

The state-of-the-art detection of TDP-43 aggregation events in cells and tissues mainly relies on protein antibodies. However, antibodies are immunogenic and can be thermally unstable (Song et al., 2012). Moreover, a single antibody cannot bind effectively all species of TDP-43 aggregates, which include oligomers in the nanometer scale as well as deposits several micrometers large. This is because TDP-43 changes its structure while aggregating and the specific portion recognized by the antibody changes the degree of exposure on the surface of the protein depending on the stage of aggregation. (Xiao et al., 2015; Gregory et al., 2020).

In addition, the average size of standard antibodies (>150-160 kDa) neither grants a fast and efficient tissue penetration, nor does it allow localization of multiple molecules on single nanometric TDP-43 aggregate that characterize the early stages of aggregation and are the most relevant to cytotoxicity. Therefore, accurate clinical identification of such early phenotypes cannot be achieved by the use of antibodies. Furthermore, TDP-43 antibodies cannot be rapidly synthetized in vitro on a large scale with low structural variation, but they require elaborate processes with high production costs, proving to be less attractive for an industrial context.

Other drug classes that are employed to bind TDP-43 aggregates are siRNA, i.e., antisense and steric blocking oligonucleotides, which however have poor intracellular uptake. Nanobodies, which are fragments of antibodies, are also employed, but while their size is much smaller than that of full-length antibodies (12-15 kDa), they show little affinity and selectivity, significant off-target effects and high production costs, without the possibility of a versatile chemical modification.

In recent years, a new class of molecules designated as “aptamers” has been tested for binding specifically to protein targets. Aptamers are chemically synthesized, single-stranded RNA or DNA oligonucleotides capable of folding into specific structures. They bind with high affinity and selectivity to a specific target molecule (in the pM/nM range) through structural recognition, similarly to protein antibodies. Due to their small size (3-15 kDa) and physico-chemical properties, aptamers provide several advantages over the aforementioned drug classes in terms of tissue penetration ability, thermal stability and solubility. They also lack immunogenicity and allow for a versatile and cost-effective synthesis processes.

The prior art discloses an RNA sequence that binds TDP-43, as reported in (Ayala et al., 2011) and (Mann et al., 2019). This sequence is 34 nucleotides in length, thus having reduced cell penetration, which is relevant for the passage through the blood brain barrier. Additionally, the literature does not disclose the affinity and specificity of this sequence to the TDP-43 protein, which makes it less attractive for experiments of binding and tracking different TDP-43 aggregate species.

In WO 2020/037234A1, a 34 nucleotide-long RNA sequence of (Ayala et al., 2011) is employed to visualise TDP-43 aggregates in cells. That 34 nucleotide-long RNA was also employed in another study (Pobran et al., 2021). This paper describes an aptamer-enrichment HPLC-MS/MS method to quantify differently spliced versions of TDP-43 in brain cells and tissue samples. However, the length of the aptamer will negatively affect its application in vivo; moreover, the presence of 8 GU repeats in the aptamer has the effect of lowering specificity, as reported in (Jolma et al., 2020). In this paper, the authors explain that many RNA-binding proteins (RBPs) function in splicing, and their motifs preferentially match sequences related to the G-U-rich splice donor sequence A/UG: GU. Indeed, proteins such as RMB38, RBM24 and HNRNPC (among others) bind to UG repeats.

Other patent applications have disclosed RNA or DNA aptamers targeting aggregation-prone proteins involved in neurodegeneration. EP3831942A1 provides a list of RNA sequences derived from known naturally-occurring binders of TDP-43, aimed at limiting the aggregation and the toxicity of TDP-43 inclusions. The inventors state that, in order to be effective, the RNA sequences must contain an uracyl (U) carrying a 2′OMe chemical modification on position 1 of the sequence and must be at least 18 nucleotides in length. Moreover, the RNA sequences described in EP3831942A1 are characterized by at least 5 GU dinucleotide units followed by an adenine.

As mentioned above, the GU dinucleotide repetitions in the sequences of EP3831942A1 are the preferential binding sites for a number of RNA-binding proteins, thus determining off-target effects. Neither binding affinity towards TDP-43 protein, nor the ability to bind TDP-43 in both the soluble and aggregated state are demonstrated in EP 3 831 942 A1.

WO 2019/032613 A1 discloses RNA and DNA sequences that are at least 20 nucleotides in length and bear many chemical modifications needed for promoting or preventing aggregation.

### SUMMARY OF THE INVENTION

An object of the present invention is to provide an aptamer capable of binding the TAR-DNA-binding protein 43 (TDP-43) with high affinity.

Another object of the present invention is to provide a TDP-43-binding aptamer characterized by fast and efficient tissue penetration and good cellular uptake.

A further object of the present invention is to provide an aptamer capable of detecting the different TDP-43 structures individually, from the soluble monomer to the larger aggregates, and which may therefore allow for an early diagnosis of the diseases involving TDP-43 aggregation.

A further object of the present invention is to provide a TDP-43-binding aptamer, which shows strong affinity and selectivity towards the TDP-43 aggregates, as well as reduced or no off-target binding (i.e., high specificity).

Still another object of the present invention is to provide a TDP-43-binding aptamer, which can be synthesized through cost-effective and versatile synthesis procedures.

Yet another object of the present invention is to provide an aptamer, which is capable of inhibiting TDP-43 aggregation and which can therefore be used for the therapeutic treatment of TDP-43-associated neurodegenerative diseases, such as ALS and FTD.

These and other objects are achieved by the isolated single-stranded TDP-43-binding DNA or RNA aptamer as defined in appended claim 1.

Further features and advantages of the single-stranded TDP-43-binding DNA or RNA aptamer of the invention will become apparent from the following detailed description of the research studies carried out by the present inventors, provided by way of illustration only, which is made with reference to the accompanying drawings.

### BRIEF DESCRIPTION OF THE DRAWINGS

FIG. 1A schematically illustrates the process of computational design of RNA aptamers through genetic algorithms that resulted in the identification of RNA aptamers of SEQ ID Nos: 1-10.

FIG. 1B is a graph showing the interaction propensity as a function of the GU content (%). RNA aptamers with strong binding propensity for TDP-43 are enriched in GU content as natural TDP-43 partners, although GU content alone does not discriminate between strong and weak interactions.

FIG. 2 is a graph showing the excellent correlation between the measured experimental affinity (nM) and the predicted Protein Fitness Score, obtained with the aptamers of SEQ ID Nos: 1-3 and 11-13.

FIG. 3 shows the results of confocal microscopy analysis of TDP-43 and Apt-1 (SEQ ID NO: 1) in mammalian cells cultures. Blue: DAPI; green: eGFP_TDP-43; red: Apt-1_Atto590. A: Wide view of Hek293T cells co-transfected with the plasmid for the overexpression of full-length TDP-43 fused to eGFP and the aptamer Apt-1 conjugated to the fluorophore Atto590; B-D: Examples of isolated cells co-transfected as in A, in which the correlation between green and red fluorescence distribution is evident; E-G: Fluorescence profiles of DAPI, eGFP and Atto590 along a diagonal line drew across the isolated correspondent cells on the left, showing corresponding distribution of the fluorescence signals of eGFP_TDP-43 and Apt-1_Atto590.

FIG. 4 shows the results of confocal microscopy analysis of TDP-43 and nApt-1 in mammalian cell cultures. Blue: DAPI; green: eGFP_TDP-43; red: Apt-1_Atto590. A: Wide view of Hek293T cells co-transfect with the plasmid for the overexpression of full-length TDP-43 fused to eGFP and the aptamer NegApt-1 conjugated to the fluorophore Atto590; B-D: Examples of isolated cells co-transfected as in A, in which the lack of correlation between green and red fluorescence distribution is evident; E-G: Fluorescence profiles of DAPI, eGFP and Atto590 along a diagonal line drew across the isolated correspondent cells on the left, showing how NegApt-1_Atto590 mostly localised in the peri-cytoplasmatic regions, irrespective of the distribution of eGFP_TDP-43.

FIG. 5 shows the results of experiments concerning the detection of pathological TDP-43 in BA4 motor cortex region of post-mortem brain sections and in post-mortem cerebrospinal fluid (CSF) with Apt-1 (SEQ ID NO:1) and its reverse complementary sequence (n-Apt-1). a) Example fluorescence Apt-1 images of post-mortem motor cortex sections from healthy control and ALS. Blue=DAPI, Red=aptamer. Apt-1 signal intensity agrees with the indicated severity of TDP-43 pathology graded by a pathologist in serial brightfield sections, scale bars: 100 μm and 50 μm; b) The number of Apt-1 detected TDP-43 aggregates increases with the severity of pathological grading of TDP-43 aggregation. c) The number of phospho-TDP43 (Ser409/410) antibody detected TDP-43 aggregates does not increase with the severity of pathological grading as strongly as with Apt-1 and shows high background in the control section. a), b) and c) show data from one sample case of each severity imaged with both probes. Individual data points in b) and c) represent mean fraction of probe positive cells in one field of view. d) Example Apt-1 and nApt-1 SR images of human post-mortem CSF taken from an ALS patient, scale bar: 1 μm. e) Using Apt-1, the number of detected TDP-43 clusters is higher in the ALS cases than with nApt-1 (p<0.0001 for case 1 and p=0.0008 for case 2, by t-test).

FIG. 6 shows the results of super-resolution imaging of TDP-43 construct aggregates using AD-PAINT. (A) Representative AD-PAINT (left) and thioflavin-T (ThT) images (right) of aggregates generated from a section of TDP-43 (RNA Recognition Motif) over time, scale bar: 500 nm. (B) Quantification of the number of aggregates detected with Apt-1 over time (mean±SD, n=3). (C) Quantification of the number of aggregates detected with ThT over time (mean±SD, n=3). (D) Quantification of the area of aggregates detected with Apt-1 over time (mean±SD, n=3) e) Intensity profile of a single oligomer. Individual localizations appear as bursts in intensity that are separated in space and time; f) Histogram of precisions of the oligomer tracked in e). Each localization is accurately positioned with a precision of 60 nm or less; g) SR image (red hot) and diffraction-limited (gray) images of the aggregate tracked in e), scale bar: 500 nm. h) Sample fields of view of clustered aggregates imaged with Apt-1 for TDP-43 (RRM1-2), α-synuclein and Amyloid-β-42, scale bar: 2 μm. i) Plot of mean cluster number per μm2 showing a significant difference in the detection of aggregates with Apt-1 and n-Apt-1 only for TDP-43. The data shown are means±SD of 9fields of view. (p<0.0001 for TDP-43 and non-significant for α-synuclein and Amyloidβ-42, by t-test).

FIG. 7 is a graph showing the aggregation kinetics of TDP-43 construct in the presence or absence of Apt-1 (SEQ ID NO:1). Blue: protein: Apt-1=1:0; Green: protein: Apt-1=1:1; Yellow: protein: Apt-1=1:2; Red: protein: Apt-1=1:4.

FIG. 8 is a graph showing the binding of the DNA version of Apt-1 (i.e. the ssDNA aptamer herein designated as Apt-14, SEQ ID NO:14) with the TDP-43 construct.

FIG. 9 shows the results of binding experiments of Apt-1 (25 nt) with the TDP-43 construct. Apt-1 (25) is a ssRNA sequence (SEQ ID NO:24) 25-nucleotides in length that contains a 8-nucleotide long protein-binding nucleotide sequence which fulfils all the features to be defined as an aptamer for the interaction with TDP-43 and which closely resembles Apt-1, except that it lacks the two 3′-terminal nucleotides. A) Binding curve of Apt-1 (25 nt) with TDP-43. A dissociation constant (K_{d}) of about 750 nM±25 was determined. B) Effect of Apt-1 (25 nt) on in vitro aggregation of TDP-43 at different protein: RNA ratios.

FIG. 10 shows the effects of a 3-fold repetition of the Apt-1 aptamer (herein designated as 3xApt-1) of in-cell aggregation of TDP-43. A) Immuno detection of soluble and insoluble TDP-43 performed with both a α-phospho TDP-43 antibody which only detects hyperphosphorylated and aggregated TDP-43, and a a-TDP-43 antibody, which detects all TDP-43 species. B) Quantification of insoluble phospho-TDP-43 (P-TDP43), insoluble eGFP-TDP-43 (eGFP-TDP43) and insoluble endogenous TDP-43 (TDP-43), in the absence and in the presence of 3xApt-1.

FIG. 11 a) shows TIRF microscope images of TDP-43 aggregates in ante-mortem CSF samples from one sporadic ALS patient and one SOD1 ALS patient detected using labelled apt-1. Left: CSF sample from a patient diagnosed with SOD1 ALS. Right: CSF sample from a patient diagnosed with sporadic ALS. FIG. 11 b) is a graph of the same experiment showing the number of detected aggregates for the sALS and the SOD1 ALS patients. Circles are results from individual technical repeats, and the error bars show the standard deviation between the points. p<0.01 (one-way ANOVA).

FIG. 12 shows standard microscopy images of formalin-fixed, paraffin-embedded post-mortem tissue taken from a control patient and a ALS patient incubated with Apt-1 and stained with the chromogen DAB.

### DETAILED DESCRIPTION OF THE INVENTION

Single-stranded RNA aptamers have been designed using new in-house developed algorithms to predict TDP-43 interactions with RNAs starting from the physico-chemical properties encoded in their sequences.

TDP-43 is a modular RNA-binding protein, whose architecture comprises an N-terminal domain, two consecutive RNA recognition motif domains (RRM1 and RRM2) that preferentially bind GU-rich RNA sequences (Zheng et al., 2018), and a low-complexity C-terminus.

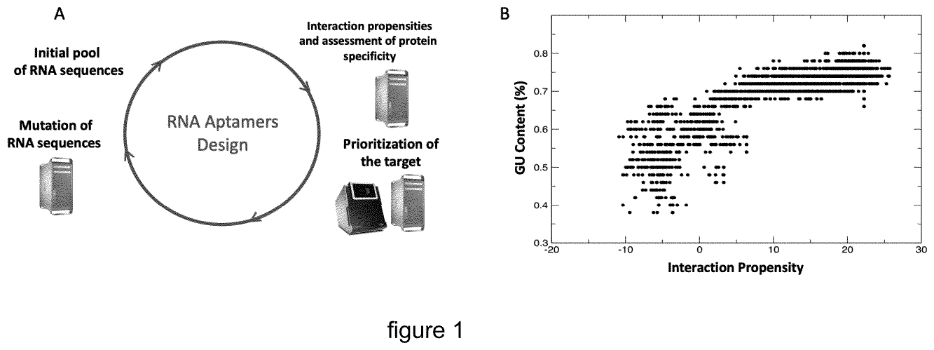

By using the aforementioned algorithms, the inventors identified single-stranded RNA molecules whose GU content increases with the binding affinity to TDP-43, thus resembling natural-like TDP-43 partners (FIG. 1B). However, the inventors also observed that the GU content is neither an indicator of specificity to TDP-43 (FIG. 1B), nor, by itself, does it strengthen the affinity towards TDP-43.

Thus, using the method schematically represented in FIG. 1A, the inventors identified the minimum sequence and structural characteristics that the protein-binding nucleotide sequence of a single-stranded RNA aptamer should possess to bind with high affinity and specificity to TDP-43. In short, the method developed by the inventors comprises selecting an initial pool of RNA sequences, which are then subjected to a number of random mutations. Thereafter, the interaction propensity and specificity for a series of proteins is estimated (Bellucci et al., 2011), among which the target of interest is prioritized (FIG. 1A).

It is known that single-stranded RNA aptamers and single-stranded DNA aptamers can be interconverted one into the other depending on the specific tasks they are used for (Amero, P. et al., 2021) and that conversion of an RNA aptamer into the corresponding DNA aptamer does not affect the TDP-43 binding properties (Kuo, P-H. et al., 2009).

Accordingly, with the method schematically represented in FIG. 1A the inventors identified a pattern of structural features which characterize the TDP-43-binding sequence of both the single-stranded RNA aptamers of the invention and their corresponding DNA versions. Such features are:
(i) the presence of at least four g nucleotides, at least two of which are consecutive, and
(ii) the presence of at least two tg/gt (DNA aptamer) or ug/gu (RNA aptamer) dinucleotides, wherein g is guanine, u is uracil and t is thymine.

In the present description, the expressions “TDP-43-binding sequence” and “protein binding sequence” indicates a stretch of the single-stranded DNA or RNA aptamer nucleotide sequence that is capable of binding the TDP-43 protein.

In the present description, the expression “at least two tg/gt dinucleotides” means that at least two tg dinucleotides, or at least two gt dinucleotides, or at least one tg dinucleotide and at least one gt dinucleotide should be present if the aptamer is a ssDNA aptamer.

Similarly, the expression “at least two ug/gu dinucleotides” means that at least two ug dinucleotides, or at least two gu dinucleotides, or at least one ug dinucleotide and at least one gu dinucleotide should be present if the aptamer is a ssRNA aptamer.

The at least two tg/gt dinucleotides or at least two ug/gu dinucleotides can be either consecutive or non-consecutive.

In a preferred embodiment, the TDP-43-binding sequence of the single-stranded DNA or RNA aptamer of the invention also includes at least one c nucleotide, wherein c is cytosine.

In a further preferred embodiment, the TDP-43-binding sequence of the single-stranded DNA or RNA aptamer of the invention is 6 to 15 nucleotides in length, preferably 8 to 10 nucleotides in length, for example 6, 7, 8, 9, 10, 11, 12, 13, 14 or 15 nucleotides in length.

In another embodiment, the single-stranded DNA or RNA aptamer of the invention contains a single TDP-43 binding sequence, in which case the full length of the aptamer is preferably between 6 to 30 nucleotides, more preferably between 8 to 25 nucleotides, even more preferably between 10 to 15 nucleotides, for example 6, 7, 8, 9, 10, 11, 12, 13, 14, 15, 16, 17, 18, 19, 20, 21, 22, 23, 24, 25, 26, 27, 28, 29 or 30 nucleotides. Alternatively, the single-stranded DNA or RNA aptamer of the invention contains multiple repeats of the TDP-43 binding sequence, preferably two or three repeats. In this embodiment, the full length of the aptamer is preferably between 12 to 90 nucleotides, more preferably between 16 to 75 nucleotides, even more preferably between 20 to 45 nucleotides, for example 12, 13, 14, 15, 16, 17, 18, 19, 20, 21, 22, 23, 24, 25, 26, 27, 28, 29, 30, 35, 40, 45, 50, 55, 60, 65, 70, 75, 80, 85 or 90 nucleotides.

In a particularly preferred embodiment, the single-stranded aptamer of the invention comprises, essentially consists or consists of the TDP-43 binding sequence of cgguguugcu (SEQ ID NO:1), gugguccccg (SEQ ID NO:2), cgcugugguc (SEQ ID NO:3), agcuguggcc (SEQ ID NO:4), cgcuggugcu (SEQ ID NO:5), cgcuguggcu (SEQ ID NO:6), cggcguuguu (SEQ ID NO:7), cgguguaggu (SEQ ID NO:8), cucuguggug (SEQ ID NO:9), or guggucgcug (SEQ ID NO:10) if the aptamer is an RNA aptamer, or the single-stranded aptamer of the invention comprises, essentially consists or consists of the TDP-43 binding sequence of cggtgttgct (SEQ ID NO:14), gtggtccccg (SEQ ID NO:15), cgctgtggtc (SEQ ID NO:16), agctgtggcc (SEQ ID NO:17), cgctggtgct (SEQ ID NO:18), cgctgtggct (SEQ ID NO:19), cggcgttgtt (SEQ ID NO:20), cggtgtaggt (SEQ ID NO:21), ctctgtggtg (SEQ ID NO:22), or gtggtcgctg (SEQ ID NO:23) if the aptamer is a DNA aptamer.

In a further preferred embodiment, the single-stranded DNA or RNA aptamer of the invention has a fluorine atom linked at position 2′ of the ribose or deoxyribose at the 3′ or 5′ end, in order to reduce degradation by nucleases.

In yet another preferred embodiment, the single-stranded DNA or RNA aptamer of the invention is labeled with a detectable label, which is more preferably selected from the group consisting of a fluorophore, a nanoparticle, a quantum dot, a nucleic acid polymer, an amino acid polymer, a hybrid nucleic acid/amino acid polymer and any combination thereof.

The affinity for TDP-43 of three of the preferred sequences mentioned above, namely SEQ ID NOs: 1, 2 and 3 (which in Table 1 below are designated as Apt-1, Apt-2 and Apt-3, respectively), was analyzed by the inventors in comparison with three other RNA sequences of the same length that do not meet the pattern of features that characterizes the single-stranded DNA or RNA aptamer of the invention. The comparison sequences are identified below as SEQ ID Nos: 11, 12 and 13 and are designated in Table 1 as Apt-4, Apt-5 and Apt-6, respectively. Table 1 shows the analyzed sequences and their structural features.

TABLE 1

| SEQ | | | | No. of GU | No. | No. of. |
|---|---|---|---|---|---|---|
| ID | | SEQUENCE | | or UG | of. | CONSECUTIVE |
| NO. | NAME | (5′ --> 3′) | LENGTH | REPEATS | G | G ≥ 2 |
| 1 | Apt-1 | CGGUGUUGCU | 10 | 3 | 4 | 1 |
| 2 | Apt-2 | GUGGUCCCCG | 10 | 2 | 4 | 1 |
| 3 | Apt-3 | CGCUGUGGUC | 10 | 3 | 4 | 1 |
| 11 | Apt-4 | GGGGUGGGGC | 10 | 1 | 8 | 2 |
| 12 | Apt-5 | CGAGGCCGGG | 10 | 0 | 6 | 2 |
| 13 | Apt-6 | GCGGGGCCCG | 10 | 0 | 6 | 1 |

In this study, the inventors calculated the predicted interaction propensities between the RNA aptamers of Table 1 and TDP-43 and validated the predicted interaction propensities by comparing them with the experimentally measured K_{d} (dissociation constant) values. To this end, the K_{d} between each of Apt-1, Apt-2, Apt-3, Apt-11, Apt-12 or Apt-13 and the two RNA recognition motifs of TDP-43 (RRM1-2) was determined by means of biolayer interferometry, a label-free technology to study biomolecular interactions. Importantly, RRM1-2 represents the minimal region necessary for RNA-binding with high affinity (Lukavsky et al., 2013) purifiable as a monomer under near-to-physiological conditions and in suitable quantities (Zacco et al., 2018). The experimental K_{d} values obtained by the inventors are provided in Table 2. Importantly, the inventors found that the experimental K_{d} values correlate with the predicted binding affinities.

Among the tested RNA sequences, Apt-1 showed the higher affinity towards RRM1-2, with a K_{d} of about 100 nM. The RNA reverse complementary sequence of Apt-1 (designated as nApt-1) was employed as a negative control. The negative control nApt-1 was found to have a K_{d} of 1.5 μM, which is comparable with the K_{d} values obtained for the worse binders Apt-11, Apt-12 and Apt-13. In contrast, Apt-1, Apt-2 and Apt-3 displayed a binding affinity for the protein TDP-43 which is comparable to the known naturally-occurring RNA binding partners, proving to be effective as diagnostic and/or therapeutic tools.

TABLE 2

| APTAMER | Kd (μM) |
|---|---|
| SEQ ID NO: 1 CGGUGUUGCU (Apt-1) | 0.10 ± 0.03 |
| SEQ ID NO: 2 GUGGUCCCCG (Apt-2) | 0.75 ± 0.15 |
| SEQ ID NO: 3 CGCUGUGGUC (Apt-3) | 0.65 ± 0.12 |
| SEQ ID NO: 11 GGGGUGGGGC (Apt-4) | 1.30 ± 0.45 |
| SEQ ID NO: 12 CGAGGCCGGG (Apt-5) | 1.50 ± 0.30 |
| SEQ ID NO: 13 GCGGGGCCCG (Apt-6) | 1.60 ± 0.40 |

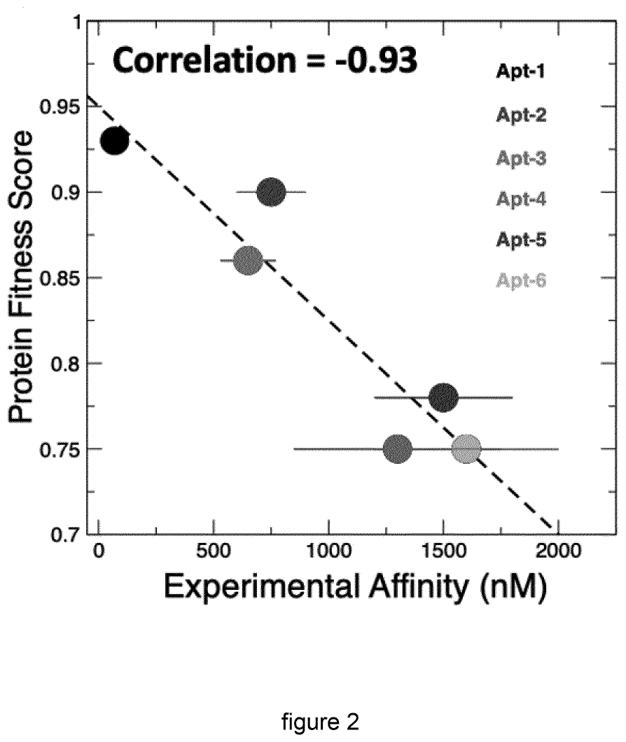

As mentioned above, the binding affinities of the tested RNA aptamers were in accordance with the scoring of the inventors' in-house algorithms (FIG. 2), indicating the high predictive power of this approach (Bellucci et al., 2011). In the calculations, the Protein Fitness score was used, which ranges between 0 and 1, to evaluate how strong is the interaction propensity of the RNA sequence for TDP-43 in comparison with a pool of protein sequences with the same length and amino acid composition (100 proteins are used for each RNA) (Agostini et al., 2013; Cirillo et al., 2013). The experimental affinity was measured as described in Example 1.

Based on the results obtained by the inventors, it is expected that further single-stranded RNA aptamers containing at least one TDP-binding sequence having the same pattern of structural features as SEQ ID NOs: 1, 2, and 3, such as for example SEQ ID NOs: 4-10, will also possess the same TDP-43-binding properties. It is also expected that single-stranded DNA aptamers containing at least one TDP-binding sequence having the same pattern of structural features as SEQ ID NOs: 1, 2, and 3, such as for example SEQ ID Nos: 14-23, will also possess the same TDP-43-binding properties.

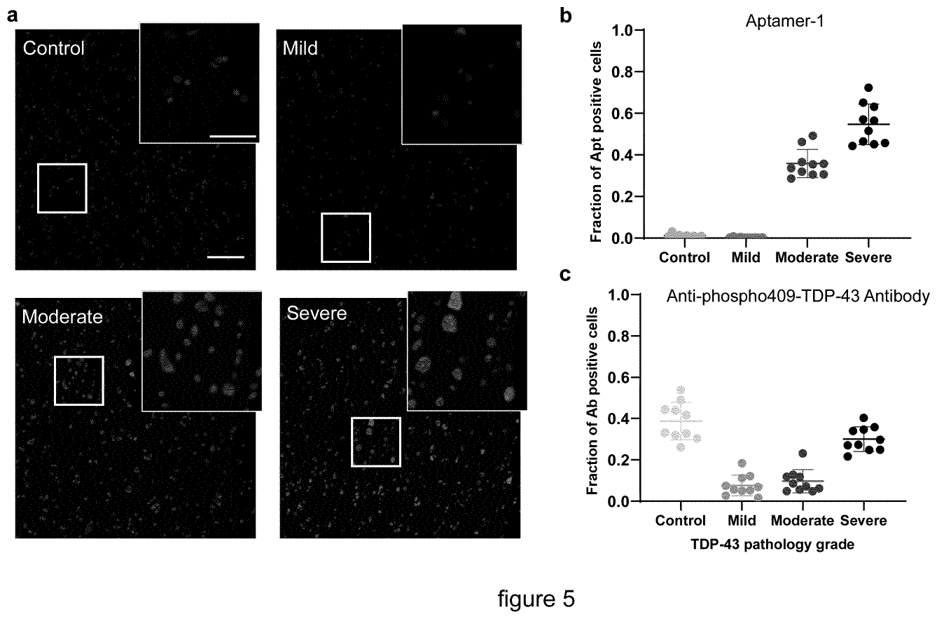

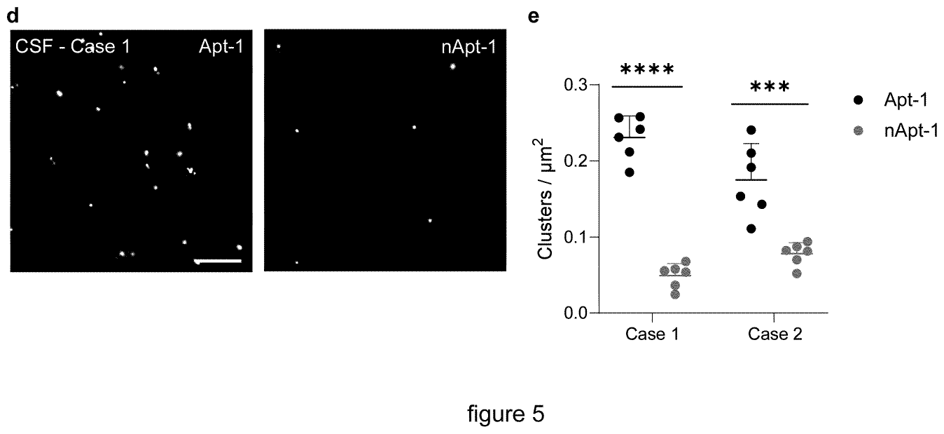

The experimental studies carried out by the present inventors, which are illustrated in detail below, showed that Apt-1 (i.e. SEQ ID NO:1) is capable of binding to and detecting TDP-43 aggregates of different sizes, from the smallest oligomers (20 nanometers) to the larger (1-2 micrometers) condensates. Apt-1 was also tested on human post-mortem tissues of ALS patients, where it proved to be an excellent detection tool for the diagnosis and/or prognosis of TDP-43-related diseases, as it was capable of distinguishing between mild, moderate and severe TDP-43 pathology (FIG. 5), according to the number of TDP-43 inclusions visualized. TDP-43-related diseases are also designated as “TDP-43 proteinopathies” and they are preferably selected from the group consisting of amyotrophic lateral sclerosis (ALS), frontotemporal lobar degeneration (FTLD), Alzheimer's disease, Lewy body dementia, Huntington's disease, argyrophilic grain dementia, Perry syndrome, progressive supranuclear palsy, corticobasal degeneration, and Pick's disease.

Just as importantly, the inventors also observed that, when added prior to the start of aggregation, Apt-1 is capable of reducing by up to 90% the rate at which solid-like TDP-43 aggregates form in solution, which makes it an excellent tool for use in the prevention and therapeutic treatment of TDP-43 proteinopathies.

Based on the results obtained by the inventors with Apt-1, it is expected that further single-stranded DNA or RNA aptamers containing at least one TDP-binding sequence having the same pattern of structural features as Apt-1, such as SEQ ID NOs: 2-10 and SEQ ID NOs: 4-10, will also possess the same TDP-43 detection and therapeutic properties.

Accordingly, further aspects of the present invention are the detection, diagnostic and therapeutic applications of the single-stranded DNA or RNA aptamers of the invention as defined in the appended claims, which form an integral part of the present description.

In particular, the single-stranded DNA or RNA aptamer of the invention is used as a detection probe for the in vitro detection of the presence, absence or amount of TDP-43 aggregates in a sample, with the aim of investigating the role of TDP-43 in various diseases in which TDP-43 aggregation occurs. In such in vitro applications, the sample can be of any type, for example a TDP-43 preparation, a biological fluid or semi-fluid or a stool sample, a human or animal cell sample, or a human or animal tissue sample. In the case of a sample taken from a subject suffering from a TDP-43 proteinopathy, the single-stranded DNA or RNA aptamer of the invention allows the distinction between an early stage and a middle/late stage of the disease, depending on the size of the TDP-43 aggregates detected. Indeed, the inventors found that the presence in the sample of TDP-43 aggregates between about 10 and 100 nm in size is indicative of an early stage of the TDP-43 proteinopathy, while the presence of aggregates between about 0.250 and 1.5 micrometers in size is indicative of a middle/late stage of the TDP-43 proteinopathy.

Due to its ability to bind all different aggregates of TDP-43, from the smallest to the largest ones, and to distinguish among them, the single-stranded DNA or RNA aptamer of the invention is also effectively used as a diagnostic agent, for the diagnosis of TDP-43 proteinopathies preferably selected from the group consisting of amyotrophic lateral sclerosis (ALS), frontotemporal lobar degeneration (FTLD), Alzheimer's disease, Lewy body dementia, Huntington's disease, argyrophilic grain dementia, Perry syndrome, progressive supranuclear palsy, corticobasal degeneration, and Pick's disease.

For the aforementioned detection and diagnostic purposes, the single-stranded DNA or RNA aptamer of the invention is preferably used in a labelled form, such as with a detectable label more preferably selected from a fluorophore, a histological staining, a biotin tag, a nanoparticle, a quantum dot, a nucleic acid polymer, an amino acid polymer, a hybrid nucleic acid/amino acid polymer and any combination thereof.

Detection of TDP-43 aggregates, either for diagnostic or research purposes, can be performed by any suitable technique, e.g. by optical microscopy detection, electron microscopy detection, electro-optic detection, electrochemical detection, mass spectroscopy analysis, biochemical detection such as size-exclusion chromatography.

Finally, because of its ability to inhibit TDP-43 aggregation, the single-stranded DNA or RNA aptamer of the invention is also effective for use in the therapeutic treatment or prevention of TDP-43 proteinopathies, such as preferably amyotrophic lateral sclerosis (ALS), frontotemporal lobar degeneration (FTLD), Alzheimer's disease, Lewy body dementia, Huntington's disease, argyrophilic grain dementia, Perry syndrome, progressive supranuclear palsy, corticobasal degeneration, and Pick's disease. For this purpose, the single-stranded DNA RNA aptamer of the invention is provided as a pharmaceutical composition comprising, in addition to the active ingredient, one or more pharmaceutically acceptable excipients, vehicles or diluents, whose selection and use is within the abilities of the person skilled in the art.

The following examples are provided by way of illustration only and are not intended to limit the scope of the invention as defined in the appended claims.

### EXAMPLES

### Example 1: Determination of the Experimental Affinity to TDP-43

The experimental affinity of SEQ ID Nos: 1-3 and 11-13 to TDP-43 was measured by biolayer interferometry experiments acquired on an Octet Red instrument (ForteBio, Inc., Menlo Park, CA) operating at 25° C. The binding assays were performed in 10 mM potassium phosphate buffer (pH 7.2) with 150 mM KCl and 0.01% Tween20. Streptavidin-coated biosensors were loaded with 1 μg/ml RNA aptamer modified with biotin on the 3′end and exposed to increasing protein concentrations varying from 20 nM to 20 μM, according to the strength of the binding. K_{d} values were estimated by fitting the response intensity (shift in the wavelength upon binding) as a function of the protein concentration, at the steady state. The assay was repeated at least 3 times, each time in triplicate. The reported binding curves are examples of the output of one experiment.

### Example 2: Analysis of Binding Ability of Aptamers Towards TDP-43 Aggregates of Different Sizes

To determine whether Apt-1 is capable of also recognizing other oligomeric species of TDP-43, the aptamer was labelled with a fluorophore and added to aggregating TDP-43 constructs in vitro at different time points. Apt-1 binds to all types of TDP-43 aggregates, from the smallest oligomers (20 nanometers) to the larger (1-2 micrometers) condensates, and it allows for the determination of the size and shape of such oligomers at different time points. The number and type of TDP-43 aggregates detected by Apt-1 was compared with the state-of-the-art aggregation detection method: thioflavin-T staining (ThT). Compared to ThT, Apt-1 recognizes a higher number of aggregates at each time point considered and detects smaller oligomers that ThT is not at all capable to intercalate. As a control for the specificity of TDP-43 towards Apt-1, the same experiments were performed employing the reverse complementary sequence of Apt-1, i.e., nApt-1. The antisense aptamer nApt-1 did not bind TDP-43 aggregates. As a control for the selectivity of Apt-1 to bind TDP-43, Apt-1 was tested on two other aggregating proteins which form similar super-molecular structures, alpha-synuclein and Amyloid-β-42. Apt-1 did not bind to alpha-synuclein or Amyloid-β-42 in any of their oligomeric forms.

### Materials and Methods

In these experiments, the immobilized protein aggregate was transiently bound by an Atto590-tagged Apt-1 molecule, the position of which was determined with nanometer precision. This process was repeated to generate an image of each aggregate. Single-molecule imaging was carried out using a custom built TIRF microscope, restricting excitation of fluorophores within the sample to 200 nm from the sample-coverslip interface. The fluorophores were excited at either 405 nm (ThT), or 561 nm (Atto590). Collimated laser light at wavelengths of 405 nm (Cobolt MLD 405-250 Diode Laser System, Cobalt, Sweden) and 561 nm (Cobolt DPL561-100 DPSS Laser System, Cobalt, Sweden) were aligned and directed parallel to the optical axis at the edge of a 1.49 NA TIRF Objective (CFI Apochromat TIRF 60XC Oil, Nikon, Japan), mounted on an inverted Nikon TI2 microscope (Nikon, Japan). A perfect-focus system corrected the imaging process for any stage-drift. Fluorescence was collected by the same objective and separated from the TIR beam by a dichroic mirror Di01-R405/488/561/635 (Semrock, Rochester, NY, USA). Collected light was then passed through appropriate filters (405 nm: BLP01-488R-25 (Semrock, NY, USA), 561 nm: LP02-568-RS, FF01-587/35 (Semrock, NY, USA). The emission beam was passed through a 2.5× beam expander and focussed onto an EMCCD camera for image collection (Delta Evolve 512, Photometrics, Tucson, AZ, USA) operating in frame transfer mode (EMGain=11.5 e⁻/ADU and 250 ADU/photon). Pixel size was 103 nm. Images were recorded with an exposure time of 50 ms with 561 nm illumination (˜50 W cm⁻¹), followed by 405 nm excitation (˜100 W cm⁻¹). The microscope was automated using the open-source microscopy platform Micromanager (NIH, Bethesda).

### Example 3: Analysis of Binding Ability of Aptamers Towards TDP-43 Aggregates of Different Sizes in Mammalian Cells Cultures

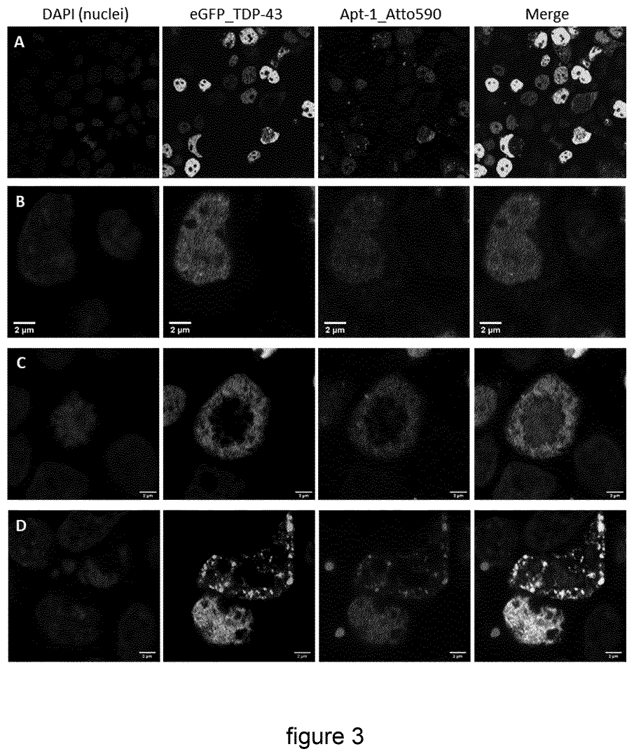

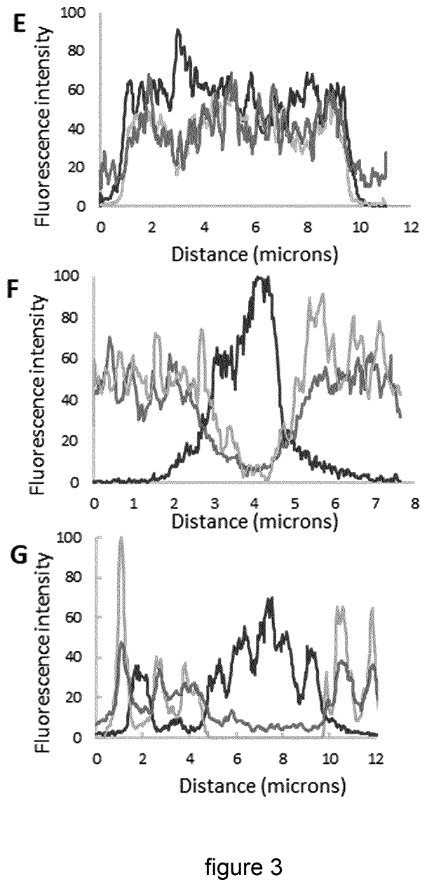

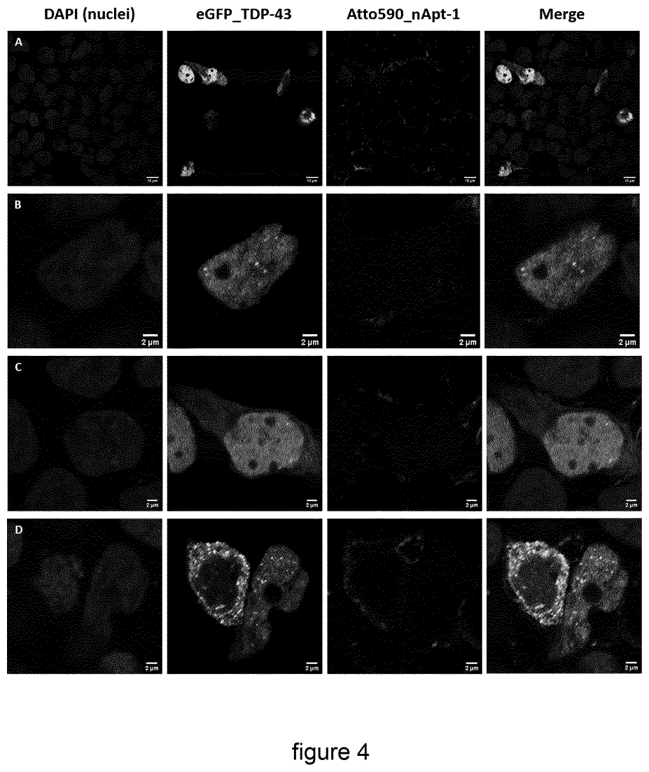

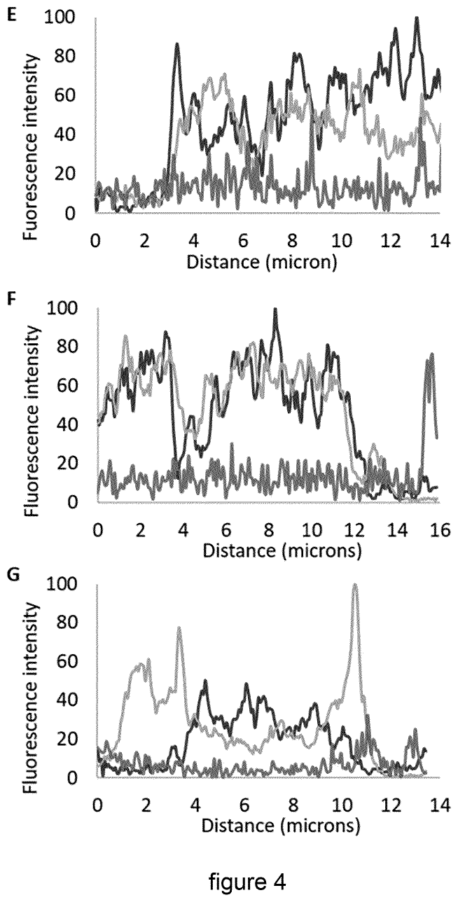

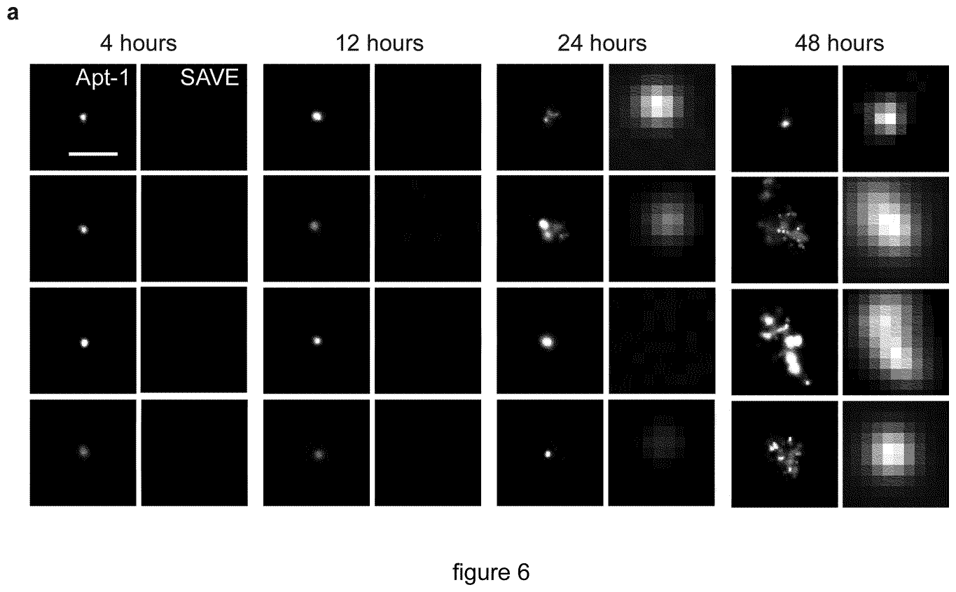

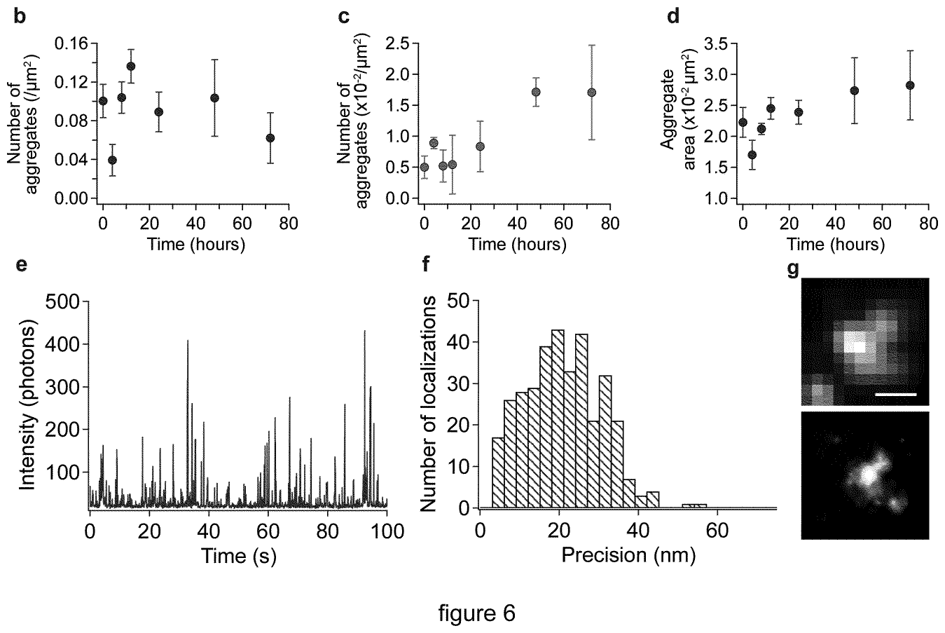

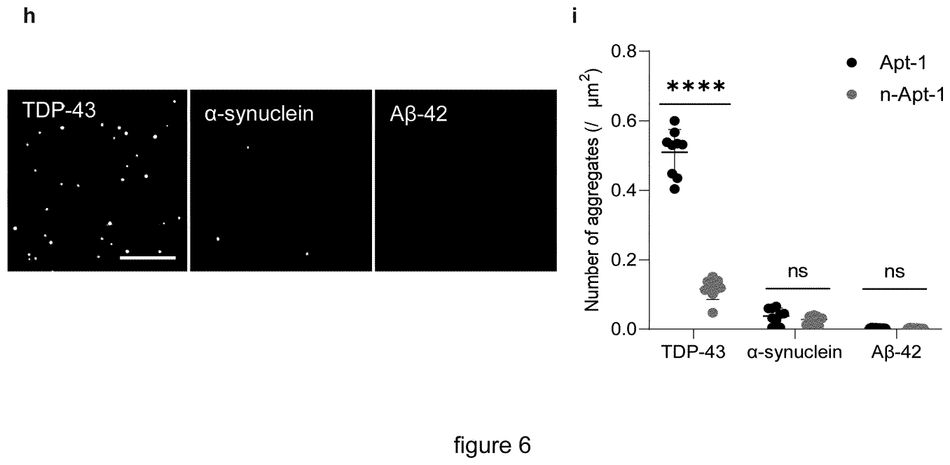

Apt-1 was tested for its ability to bind a range of TDP-43 species in cells. This would potentially offer great advantage in the use of RNA aptamers as analytical and diagnostic tools. Full-length TDP-43 fused with eGFP was expressed in Hek 293T concomitantly to Apt-1 conjugated to the fluorophore Atto590 at the 3′ end for easier detection. To reduce nuclease degradation, the cytosines at positions 1 and 9 of Apt-1 were chemically modified with the 2′-fluoro modification. After 24 hours, the cells were fixed and visualized by confocal microscopy (FIG. 3). Following TDP-43 distribution, cells were found in a mixed population composed of healthy elements and cells with mislocalized and/or aggregated TDP-43, which recapitulate the ALS phenotype (FIG. 3a). Zooming in on these cells, the sub-cellular distribution of TDP-43 and Apt-1 in both healthy and phenotypically unhealthy cells was analyzed by imaging (FIG. 3b-d) and fluorescence profiling (FIG. 3e-g), in which the fluorescence intensity of DAPI (nuclei), eGFP (TDP-43) and Atto590 (Apt-1) was followed, pixel by pixel. Healthy cells showed a nuclear distribution of both TDP-43 and Apt-1 (FIG. 3b), confirming the interaction between soluble TDP-43 and the aptamer. The result was supported by the fluorescence profile, in which intensities of DAPI, eGFP and Atto590 correlate (FIG. 3e). The unhealthy cells, either with homogenous cytosolic distribution of TDP-43 (FIG. 3c) or with cytosolic TDP-43 aggregates (FIG. 3d) showed both co-localisation and correlation between fluorescence profiles of TDP-43 and Apt-1 (FIG. 4f-g). For these cells, the co-localization of Apt-1 and TDP-43 protein was quantified by measuring the Manders' overlap, which determines the co-occurrence of the two fluorescence signals while taking into account pixel intensity in a Z-stack (Manders et al., 1993). For the cells shown in FIG. 3c, Manders' overlap was 0.85/1, while for the cells shown in FIG. 3D it was 0.89/1. These values indicate a co-localization of the fluorescence associated with TDP-43 and Apt-1 of 85% and 89%, respectively. The same experiments were performed with the antisense control nApt-1, which instead did not co-localise nor it correlated with TDP-43 (FIG. 5a), be it soluble or aggregates, as demonstrated by confocal imaging (FIG. 5b-d), fluorescence profiling (FIG. 6e-g) and Mander's overlap, which for the cells reported had an average value of 0.2/1. These analyses confirmed that Apt-1 can tightly bind to any oligomerization state of full-length TDP-43 within the context of live cells and suggested that aptamers designed with the inventors' in-house algorithms can be employed as probes for the visualization and identification of their intracellular targets.

### Materials and Methods

In these experiments, human embryonic kidney (HEK) 293T cells were cultured in Dulbecco's modified eagle medium (DMEM) enriched with L-glutamine and kept at 37° C. with 5% CO₂. For microscopy, cells were plated on 24-well plates containing coverslips pre-treated with poly-L-lysine. After 24 hours, or when confluence was around 65%, cells were co-transfected with 1.5 μg/ml DNA plasmid for TDP-43 overexpression and 1 μg/ml RNA aptamer using the transfection agent Lipofectamine 3000, according to the published protocol (Invitrogen). TDP-43 wild-type gene was cloned downstream to the eGFP gene in a pEGFP C1 mammalian transfection vector; Apt-1 was purchased with the fluorophore Atto590 at its 3′ end and with the cytosines at positions 1 and 9 were chemically modified with 2′-fluoro modification, to increase in-cell stability against nuclease degradation. nApt-1 was purchased with the fluorophore Atto590 at its 5′ end and with the guanines at positions 1 and 9 chemically modified with 2′-fluoro modification, to increase in-cell stability against nuclease degradation. 24 hours after transfection, cells were washed with phosphate-buffered saline (PBS) solution and fixed with 4% paraformaldehyde for 10 minutes at room temperature. After further washes in PBS, cells were permeabilised with 0.1% Triton-X 100in PBS for 3 minutes, washed and treated with 0.5 μg/ml 4′,6-diamidino-2-phenylindole (DAPI) solution. After further washing in PBS, coverslips were placed faced down on glass slides using the mounting medium ProLong™ Diamond Antifade Mountant (Invitrogen). Glass slides with fixed cells were visualized with a Nikon's A1R MP multiphoton confocal microscope, employing the 60× objective and 3 channel non-descanned detectors. Acquisition and analysis were performed with the Nikon software NIS-Elements Advanced Research version 5.30.02, 64 bit. Fluorescence profiles were defined by drawing a line across specific cells and calculating the fluorescence intensity of the three fluorophores (DAPI, eGFP and Atto590) pixel by pixel. Z-stacks for selected cells were composed acquiring scans every 0.5 μm for 6 μm above and below the median plane. Through the Z-stacks, the Manders' overlap, which determines the co-occurrence of two selected fluorescence signals while taking into account pixel intensity, was derived by exploring the correlation between green and red fluorescence values. Cells' 3D images were reconstructed from the Z-stacks. Transfection and images acquisitions were repeated 3 times, each time in duplicates. Fluorescence profiling and Mander's overlap was calculated for at least 25 cells per sample.

### Example 4: Analysis of Binding and Detection Ability of Aptamers Towards TDP-43 Aggregates of Different Sizes in Human Post-Mortem CSF and Tissue of ALS Patients with Different Severity Grades of TDP-43 Pathology (Mild, Moderate and Severe) and Comparison With Commercially Available TDP-43 Antibodies

The aptamer, Apt-1, was used to image TDP-43 in ALS brain tissue. The number of TDP-43 inclusions detected with Apt-1 increased with the severity of TDP-43 aggregation as assessed using gold-standard immunohistochemistry by a neuropathologist. The use of Apt-1 allowed differentiation between mild, moderate and severe TDP-43 pathology (FIG. 5). Apt-1 also allowed SR detection, counting and sizing of aggregates in CSF, proving it to be an excellent detection tool for the diagnosis and/or prognosis of TDP-43 diseases. On the contrary, the commercially available antibody shows high signal in control samples and cannot easily differentiate between the different severities of TDP-43 pathology in fluorescence imaging.

### Materials and Methods

In these experiments, standard fluorescence microscopy was employed to image TDP-43 in the BA4 motor cortex region of post-mortem brain tissue. A commercially available antibody (Phospho-TDP43 (Ser409/410) Polyclonal antibody, Proteintech) was used for comparison with Apt-1. For details on the microscopy, see Example 2.

### Example 5: Analysis of Binding Ability of Aptamers Towards TDP-43 Aggregates of Different Size in Human Post-Mortem Tissue of ALS Patients and Comparison with Commercially Available Dyes

The aptamer Apt-1 was linked to a fluorescent probe for the visualization by super-resolution microscopy (AD-PAINT). FIG. 6 shows an improved detection of misfolded TDP-43 protein species compared to a standard visualization method based on the commercially available ThT dye. The use of Apt-1 as imaging probe allows the analysis of the progression of TDP-43 aggregation, especially the early stages that are considered more relevant to cytotoxicity and clinical phenotypes, and enables size determination at the nanometer scale, contrary to standard detection methods.

### Materials and Methods

In these experiments, Atto-590 tagged Apt-1 was diluted in PBS buffer and used at a final imaging concentration of 1 nM. ThT (Sigma) was dissolved in absolute ethanol (99%) and then diluted in PBS buffer and filtered (0.02 μm filtered, Anotop25, Whatman). The exact concentration was determined by absorbance (8421 nm=36 000 M⁻¹ cm⁻¹). The ThT solution was further diluted to 5 μM in the solution of Atto590-tagged Apt-1 for AD-PAINT imaging. Positions of the transiently immobilised Apt-1 within each frame were determined using the PeakFit plugin (an imageJ/Fiji plugin of the GDSC Single Molecule Light Microscopy package (http://www.sussex.ac.uk/gdsc/intranet/microscopy/imagej/gdsc_plugins) for imageJ using a “signal strength” threshold of 30 and a precision threshold of 60 nm. The localizations were grouped into clusters using the DBSCAN algorithm in Python 3.8 (sklearn v0.24.2) using epsilon=1 pixels and a minimum points threshold of 30 to remove random localizations. The clustered localizations were plotted as 2D Gaussian distributions, with a width equal to the precision that they were localized to. To determine the length and areas of each cluster, the localizations were plotted with widths equal to the precision FWHM and were then analysed using measure module (skimage v0.18.1). The lengths quoted are the maximum measured axis distance. The SAVE images were first thresholded using a value of intensity mean+2×S.D., and then analysed using the measure module (skimage v0.18.1). Aggregate clusters in the AD-PAINT images were to be ThT-active if any of the localizations had ThT signal greater than the threshold value in the corresponding SAVE image. For each sample, three fields of view were imaged.

### Example 6: Analysis of the Ability of Aptamers of Inhibiting TDP-43 Aggregation

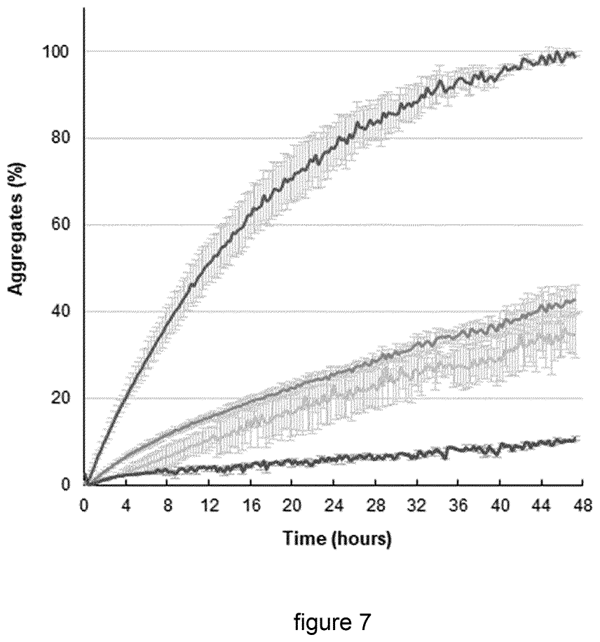

TDP-43 aggregation was monitored in the presence of Apt-1 in the test tube. The inventors observed that, when added prior to the start of aggregation, Apt-1 is capable of reducing by up to 90% the rate at which solid-like TDP-43 aggregates form in solution (FIG. 7).

Materials and Methods

In these experiments, purified protein samples stored at −80° C. were rapidly defrosted and diluted to 20 μM in a high salt buffer (10 mM potassium phosphate buffer pH 7.2, 150 mM KCl). Constructs were subsequently spun at 100,000 g for 1 h to remove any degraded or aggregated protein. The final protein concentration was assessed by absorbance at 280 nm and adjusted to 15 μM. Protein aggregation was carried out at 37° C. under non-shaking conditions. Aliquots were taken at given time points (0, 4, 8, 12, 24, 48, 72 h) and flash frozen for later analysis. The experiments were repeated three times.

The experimental results obtained by the inventors in Examples 2-6 confirm that Apt-1 is able to prevent TDP-43 aggregation and to abrogate toxic TDP-43 aggregation, thereby increasing cell viability. They also demonstrate that Apt-1 overcomes the limitations of the prior art, affording a binding with TDP-43 protein with greater affinity and specificity in every aggregation state, a great stability in the biological environment and ease of production (even on an industrial scale), proving to be an excellent tool in detection, diagnostic and therapeutic applications.

Examples 2-6 have been carried out with the RNA aptamer Apt-1, consisting of SEQ ID NO: 1. Given the high predictive power of the inventors' designing tool, it is plausible that ssRNA aptamers sharing the same structural features as SEQ ID NO:1 shall provide similar results. Plausibility is supported by Examples 8 and 9 below, which disclose experiments carried out with a 25-nt long ssRNA aptamer that contains an 8-nt long TDP-43-binding nucleotide sequence having all the features identified in appended claim 1, and with a 30-nt long ssRNA aptamer consisting of a 3-fold repetition of Apt-1, respectively.

Considering that KUO P-H et al, 2009 described that a requirement for nucleotide sequences to bind TDP-43 is the presence of TG/UG motifs, and also considering that the same paper further acknowledged that both RRM domains of TDP-43 (i.e. RRM1 and RRM2) participate in binding to both DNA and RNA sequences, it is also plausible that ssDNA aptamers having the same features defined in appended claim 1 shall provide similar results as those obtained with Examples 2-6. Plausibility is supported by Example 7 below, which discloses binding experiments carried out with the DNA version of Apt-1 (i.e., with the ssDNA sequence designated as Apt-14), in which a dissociation constant (K_{d}) of about 76 nM was determined, which is fully comparable with the dissociation constant determined with Apt-1 (K_{d} of about 90 nM).

### Example 7: Apt-14 Still Maintains Tight Binding Towards TDP-43

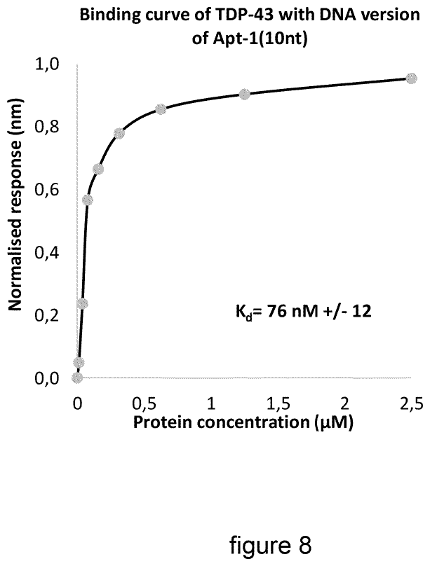

The inventors verified whether Apt-14 preserves the ability to bind tightly to TDP-43. Bio-layer interferometry was used to determine the dissociation constant of the interaction between Apt-14 and the TDP-43 construct. This experiment was carried out with the same buffer and the same conditions employed for the experiment carried out with Apt-1. A K_{d} of about 76 nM was determined (see FIG. 8), comparable to the one determined for Apt-1 (about 90 nM).

### Example 8: Apt-1 (25nt) is Able to Tightly Bind to TDP-43 and to Reduce in Vitro TDP-43 Aggregation

The aim of this study was to test whether the 10-nucleotide ssRNA aptamer designated as Apt-1 is able to preserve its binding affinity and anti-aggregation properties also when inserted in a longer sequence. The following 25-nt RNA sequence was employed: 5′-GCUGGGGUGGGGCGGAUCGGUGUUG-3′ (SEQ ID NO:24, Apt-1 (25)). Apt-1 (25 nt) contains 8 of the 10 nucleotides of Apt-1 (marked in bold). These 8 nucleotides fulfil the features defined in appended claim 1 for the interaction with TDP-43, namely the presence of at least two GU/UG; at least four G, two of which consecutive; and at least 1 C.

Bio-layer interferometry analysis was performed to determine the binding affinity of Apt-1 (25 nt) for the TDP43 construct and a K_{d} of about 750 nM was determined, which is not as tight as that of Apt-1 but still in the optimal range of >1 μM.

The inventors also determined the effect of Apt1 (25 nt) on the in vitro aggregation of the TDP-43 construct. The aggregation process was followed with a fluorescent dye and the aggregate content was determined as a function of time. This assay showed that Apt-1 (25nt) is able to reduce TDP-43 aggregation up to about 70%.

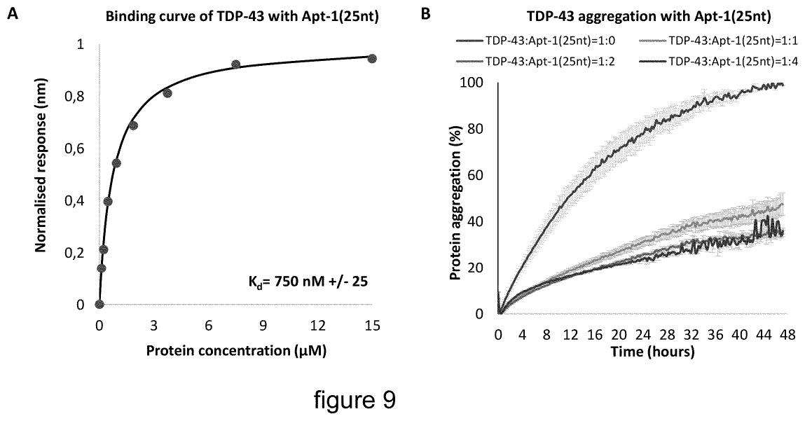

The results of this study are illustrated in FIGS. 9A and 9B.

### Example 9: Concatenations of Apt-1 are Able to Reduce Aggregated TDP-43 in Mammalian Cells

This study was aimed at verifying the effect of aptamer concatenation on TDP-43 aggregation. A 3-fold concatenation of the RNA aptamer Apt-1 (designated herein as 3xApt-1) was employed. The effect of 3xApt-1 on TDP-43 aggregation in a mammalian cell model was quantitatively determined. HEK 293T cells were transiently transfected with a vector over-expressing TDP-43 fused with eGFP, using a commercially available lipophilic transfection reagent. At the same time, the cells were also given increasing concentrations of 3xApt-1 (0.5-2 μg/ml). The cells were collected after 48 hours and the total protein content was extracted by lysis in RIPA buffer, followed by a short sonication step and by centrifugation. The supernatant collected after the centrifugation represented the soluble protein fraction. The pellet—insoluble protein fraction—was resuspended in 7 M urea with 10% SDS and left under agitation in this solution for 1 hour. The extracted proteins were quantified from the soluble fraction and an equal volume of soluble and insoluble proteins was loaded on a SDS-PAGE, so as to compare side by side the proteins extracted from the cells over-expressing TDP-43-eGFP only with the proteins extracted from cells co-transfected with TDP-43-eGFP and 3xApt-1. The gel was transferred on a nitrocellulose membrane and 24ecogn-stained with two different antibodies: an antibody against phospholylated-TDP-43, recognizing solely the hyperphosphorylated aggregated TDP-43,and an antibody against all forms of TDP-43. Since the cells were transfected with TDP-43-eGFP, we expected to identify both endogenous TDP-43 and transfected TDP-43-eGFP. The quantification of the different species of TDP-43 identified with both antibodies, performed with the software ImageJ, demonstrates a reduction in the aggregated, insoluble TDP-43 form by 20%-50%, according to the specie.

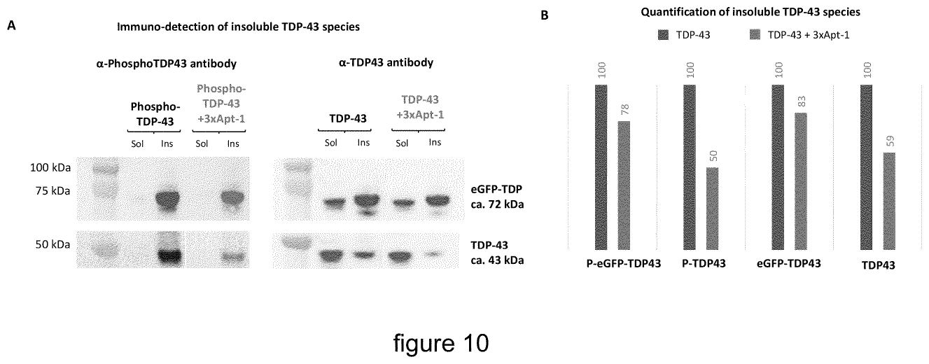

The results of this study are illustrated in FIGS. 10A and 10B.

### Example 10: Detection of TDP-43 Aggregates in Ante-Mortem CSF Samples from ALS Patients

CSF was collected from one sporadic ALS patient and one SODI ALS patient. 20 μL of each CSF sample was diluted 2-fold into a solution containing 200 nM apt-1 (labelled with Atto590) and 8% PFA. Following incubation overnight, the samples were diluted 10-fold into PBS, and incubated on a plasma-cleaned glass coverslip for 30 minutes. The samples were subsequently imaged on a home-built total internal reflection fluorescence (TIRF) microscope. The TIRF microscope uses collimated laser light at a wavelength of 561 nm (Cobolt DPL561-100 DPSS Laser System, Cobalt, Sweden) aligned and directed parallel to the optical axis at the edge of a 1.49 NA TIRF Objective (CFI Apochromat TIRF 60XC Oil, Nikon, Japan), mounted on an inverted Nikon TI2 microscope (Nikon, Japan). A perfect-focus system corrected the imaging process for any stage-drift. Fluorescence was collected by the same objective and separated from the TIR beam by a dichroic mirror Di01-R405/488/561/635 (Semrock, Rochester, NY, USA). Collected light was then passed through appropriate filters (LP02-568-RS, FF01-587/35 (Semrock, NY, USA)). The emission beam was passed through a 2.5× beam expander and focused onto an EMCCD camera for image collection (Delta Evolve 512, Photometrics, Tucson, AZ, USA) operating in frame transfer mode (EMGain=11.5 e-/ADU and 250 ADU/photon). Pixel size was 103 nm. Images were recorded for 50 frames with an exposure time of 50 ms with 561 nm illumination (˜50 W cm⁻1). The microscope was automated using the open-source microscopy platform Micromanager (NIH, Bethesda). The analysis was performed using ImageJ. Following average z-projection over the 50 frames, the “spot-count” function was used (with a noise-tolerance of 100) to count the number of spots per field-of-view.

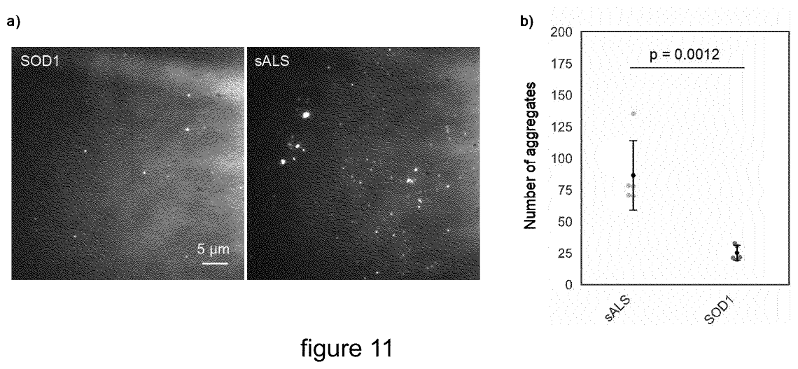

The results of the study are illustrated in FIGS. 11 a) and 11 b).

### Example 11: Histological Staining and Standard Microscopy

Human spinal cord tissue from a control and a ALS case was taken at post-mortem and fixed in 10% neutral-buffered formalin for a minimum of 72 hours. The tissue was dehydrated in an ascending alcohol series (70-100%), followed by three 4-h xylene washes. Three successive 5-h paraffin wax embedding stages were performed, followed by cooling and sectioning of formalin-fixed, paraffin-embedded (FFPE) tissue on a Leica microtome into 4-μm-thick serial sections, collected on Superfrost slides. Sections were dried overnight at 40° C. and dewaxed using successive xylene washes, followed by alcohol hydration and treatment with picric acid (15 minutes) to minimise formalin pigment. Antigen retrieval was performed in citric acid buffer (pH 6) in a pressure cooker for 30 min. Following antigen retrieval, slides were washed once with distilled water for 5 minutes followed by incubation with peroxidase block (Leica kit) for 30 minutes followed by a wash step in TBS for 5 minutes and incubation in protein block (Leica kit) for 15 minutes followed by a wash step in TBS for 5 minutes. In the present description, “Leica kit” indicates the following reagents: Novolink Polymer detection system (Leica Biosystems). The TDP-43 RNA aptamer (Apt-1) was then diluted 1 in 500 in TBS and incubated on the slides for 5 hours at 4° C. Then, the slides were incubated overnight at 4° C. in PFA (with no intervening wash step). Following overnight incubation, the slides were washed in distilled water for 5 minutes and incubated with an anti-biotin antibody conjugated to HRP (Abcam; Ab6651 at 1 in 100 dilution in TBS) for 30 minutes at room temperature followed by a 5 minute wash in distilled water and incubation with DAB for 5 minutes. The slides were then washed carefully in running tap water and counterstained using haematoxylin (2 minutes) and blued with lithium carbonate (30 s) and mounted with DPX.

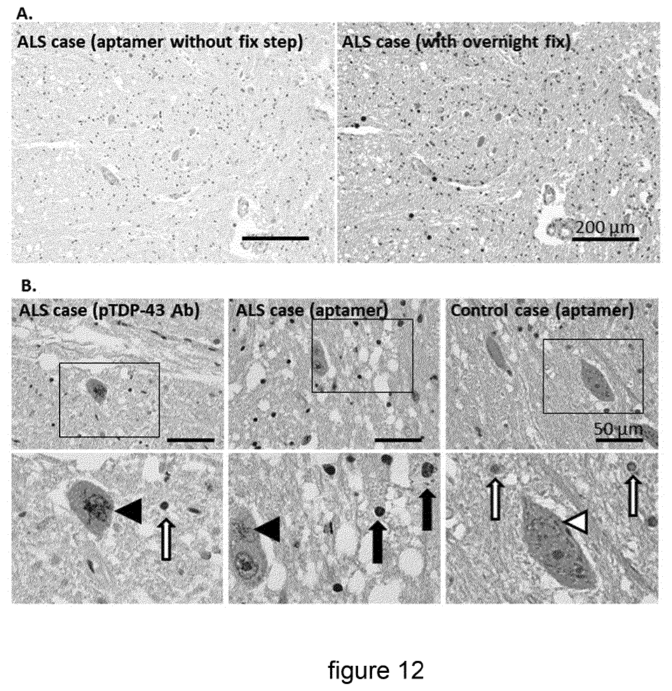

FIG. 12 shows standard microscopy images of this experiment. A. Serial sections show the same region of the anterior horn of human spinal cord from a ALS patient. The image on the left shows staining when no fixation step is performed (no evidence of brown DAB chromogen, i.e., no evidence of detection of the TDP-43 pathology). The image on the right shows extensive DAB chromogen deposition, i.e., detection of extensive TDP-43 pathology. B. Serial sections show the same region of the anterior horn of human spinal cord from a ALS patient (left and central images) and the anterior horn of an age-and sex matched control case that has no TDP-43 pathology and does not have ALS. The top panel shows lower power image and lower panel shows zoomed in area highlighted by the box in the upper panel. Arrowheads indicate neurons showing cytoplasmic neuronal TDP-43 aggregation (black arrowhead) in the ALS cases, but not in the control case (white arrowhead). Arrows indicate glial cells with glial TDP-43 aggregation only seen with the aptamer (black arrow) and no TDP-43 detected in the control case nor in the ALS case using the pTDP-43 antibody (white arrow). These data highlight the improved sensitivity and specificity of the aptamer compared to conventional antibody approaches and demonstrates the necessity of the fixation step.

### REFERENCES

Agostini, F., Cirillo, D., Bolognesi, B., and Tartaglia, G.G. (2013). X-inactivation: quantitative predictions of protein interactions in the Xist network. Nucleic Acids Research 41, e31.
Amero, P., Lokesh, G., Chaudhari, R. R., Cardenas-Zuniga, R., Schubert, T., Attia, Y. M., Montalvo-Gonzalez, E., Elsayed, A. M., Ivan, C., Wang, Z., Cristini, V., Franciscis, V., Zhang, S., Volk, D. E., Mitra, R., Rodriguez-Aguayo, C., Sood, A. K., & Lopez-Berestein, G. (2021). Conversion of RNA Aptamer into Modified DNA Aptamers Provides for Prolonged Stability and Enhanced Antitumor Activity. Journal of the American Chemical Society, 143 (20), 7655-7670.
Ayala, Y.M., De Conti, L., Avendaño-Vázquez, S.E., Dhir, A., Romano, M., D'Ambrogio, A., Tollervey, J., Ule, J., Baralle, M., Buratti, E., et al. (2011). TDP-43 regulates its mRNA levels through a negative feedback loop. EMBO J. 30, 277-288.
Bellucci, M., Agostini, F., Masin, M., and Tartaglia, G.G. (2011). Predicting protein associations with long noncoding RNAs. Nat. Methods 8, 444-445.
Cirillo, D., Agostini, F., Klus, P., Marchese, D., Rodriguez, S., Bolognesi, B., and Tartaglia, G.G. (2013). Neurodegenerative diseases: Quantitative predictions of protein-RNA interactions. RNA 19, 129-140.
Gregory, J.M., McDade, K., Bak, T.H., Pal, S., Chandran, S., Smith, C., and Abrahams, S. (2020). Executive, language and fluency dysfunction are markers of localised TDP-43 cerebral pathology in non-demented ALS. J Neurol Neurosurg Psychiatry 91, 149-157.
Jolma, A., Zhang, J., Mondragón, E., Morgunova, E., Kivioja, T., Laverty, K.U., Yin, Y., Zhu, F., Bourenkov, G., Morris, Q., et al. (2020). Binding specificities of human RNA-binding proteins toward structured and linear RNA sequences. Genome Res 30, 962-973.
Kuo, P. H., Doudeva, L. G., Wang, Y. T., Shen, C. K., & Yuan, H. S. (2009). Structural insights into TDP-43 in nucleic-acid binding and domain interactions. Nucleic acids research,37 (6), 1799-1808.
Lukavsky, P.J., Daujotyte, D., Tollervey, J.R., Ule, J., Stuani, C., Buratti, E., Baralle, F.E., Damberger, F.F., and Allain, F.H.-T. (2013). Molecular basis of UG-rich RNA recognition by the human splicing factor TDP-43. Nat Struct Mol Biol 20, 1443-1449.
Manders, E.M.M., Verbeek, F.J., and Aten, J.A. (1993). Measurement of co-localization of objects in dual-colour confocal images. J Microsc 169, 375-382.
Mann, J.R., Gleixner, A.M., Mauna, J.C., Gomes, E., DeChellis-Marks, M.R., Needham, P.G., Copley, K.E., Hurtle, B., Portz, B., Pyles, N.J., et al. (2019). RNA Binding Antagonizes Neurotoxic Phase Transitions of TDP-43. Neuron 102, 321-338.e8.
Marchese, D., Botta-Orfila, T., Cirillo, D., Rodriguez, J.A., Livi, C.M., Fernández-Santiago, R., Ezquerra, M., Martí, M.J., Bechara, E., and Tartaglia, À. (2017). Discovering the 3′ UTR-mediated regulation of alpha-synuclein. Nucleic Acids Res 45, 12888-12903.
Pobran, T.D., Yang, D., Mackenzie, I.R.A., and DeMarco, M.L. (2021). Aptamer-based enrichment of TDP-43 from human cells and tissues with quantification by HPLC-MS/MS. J Neurosci Methods 363, 109344.
Ratti, A., and Buratti, E. (2016). Physiological functions and pathobiology of TDP-43 and FUS/TLS proteins. J Neurochem 138 Suppl 1, 95-111.
Song, K.-M., Lee, S., and Ban, C. (2012). Aptamers and Their Biological Applications. Sensors (Basel) 12, 612-631.
Tollervey, J.R., Curk, T., Rogelj, B., Briese, M., Cereda, M., Kayikci, M., König, J., Hortobágyi, T., Nishimura, A.L., Župunski, V., et al. (2011). Characterizing the RNA targets and position-dependent splicing regulation by TDP-43. Nat Neurosci 14, 452-458.
Xiao, S., Sanelli, T., Chiang, H., Sun, Y., Chakrabartty, A., Keith, J., Rogaeva, E., Zinman, L., and Robertson, J. (2015). Low molecular weight species of TDP-43 generated by abnormal splicing form inclusions in amyotrophic lateral sclerosis and result in motor neuron death. Acta Neuropathol 130, 49-61.
Zacco, E., Martin, S.R., Thorogate, R., and Pastore, A. (2018). The RNA-Recognition Motifs of TAR DNA-Binding Protein 43 May Play a Role in the Aberrant Self-Assembly of the Protein. Front Mol Neurosci 11, 372.

1. An isolated single-stranded RNA or DNA aptamer, which comprises a protein-binding nucleotide sequence capable of binding a RNA recognition motif (RRM) of the TDP-43 protein, wherein the protein-binding nucleotide sequence includes: (i) at least four g nucleotides, at least two of which are consecutive, and (ii) at least two tg/gt dinucleotides or at least two ug/gu dinucleotides, wherein g is guanine, u is uracil and t is thymine.

2. The isolated single-stranded RNA or DNA aptamer according to claim 1, wherein the protein-binding nucleotide sequence includes at least one c nucleotide, wherein c is cytosine.

3. The isolated single-stranded RNA or DNA aptamer according to claim 1, wherein the protein-binding nucleotide sequence is 6 to 15 nucleotides in length.

4. The isolated single-stranded RNA or DNA aptamer according to claim 1, wherein the aptamer is a single-stranded RNA aptamer and the protein-binding nucleotide sequence is selected from the group consisting of cgguguugcu (SEQ ID NO: 1), gugguccccg (SEQ ID NO:2), cgcugugguc (SEQ ID NO:3), agcuguggcc (SEQ ID NO:4), egcuggugcu (SEQ ID NO:5), cgcuguggcu (SEQ ID NO:6), eggeguuguu (SEQ ID NO:7), cgguguaggu (SEQ ID NO:8), cucuguggug (SEQ ID NO:9), or guggucgcug (SEQ ID NO: 10).

5. The isolated single-stranded RNA or DNA aptamer according to claim 1, wherein the aptamer is a single-stranded DNA aptamer and the protein-binding nucleotide sequence is selected from the group consisting of eggtgttgct (SEQ ID NO: 14), gtggtccccg (SEQ ID NO: 15), cgctgtggtc (SEQ ID NO: 16), agetgtggcc (SEQ ID NO: 17), cgctggtgct (SEQ ID NO: 18), cgctgtgget (SEQ ID NO: 19), eggegttgtt (SEQ ID NO:20), cggtgtaggt (SEQ ID NO:21), ctctgtggtg (SEQ ID NO:22), or gtggtegctg (SEQ ID NO:23).

6. The isolated single-stranded RNA or DNA aptamer according to claim 5, which is 10 to 15 nucleotides in length.

7. The isolated single-stranded RNA or DNA aptamer according to claim 6, wherein the aptamer is a single-stranded RNA aptamer and consists of the protein-binding nucleotide sequence selected from the group consisting of egguguugcu (SEQ ID NO:1), gugguccecg (SEQ ID NO: 2), cgcugugguc (SEQ ID NO:3), agcuguggcc (SEQ ID NO:4), cgcuggugcu (SEQ ID NO: 5), cgcuguggcu (SEQ ID NO:6), eggeguuguu (SEQ ID NO:7), cgguguaggu (SEQ ID NO:8), cucuguggug (SEQ ID NO:9), or guggucgcug (SEQ ID NO: 10).

8. The isolated single-stranded RNA or DNA aptamer according to claim 6, wherein the aptamer is a single-stranded DNA aptamer and consists of the protein-binding nucleotide sequence selected from the group consisting of cggtgttget (SEQ ID NO: 14), gtggtecccg (SEQ ID NO: 15), cgctgtggtc (SEQ ID NO: 16), agetgtggcc (SEQ ID NO: 17), cgetggtget (SEQ ID NO:

18., cgctgtggct (SEQ ID NO: 19), cggcgttgtt (SEQ ID NO:20), cggtgtaggt (SEQ ID NO:21), ctctgtggtg (SEQ ID NO:22), or gtggtegetg (SEQ ID NO:23).

9. The isolated single-stranded RNA or DNA aptamer according to claim 1, which comprises multiple repeats of the protein-binding nucleotide sequence, preferably two or three repeats.

10. The isolated single-stranded RNA or DNA aptamer according to claim 1, wherein the ribose or deoxyribose at the 3′ or 5′ end has a fluorine atom linked at the 2′ position.

11. The isolated single-stranded RNA or DNA aptamer according to claim 1, which is labeled with a detectable label.

12. The isolated single-stranded RNA or DNA aptamer according to claim 11, wherein the detectable label is selected from the group consisting of a fluorophore, a histological staining, a biotin tag, a nanoparticle, a quantum dot, a nucleic acid polymer, an amino acid polymer, a hybrid nucleic acid/amino acid polymer and any combination thereof.

13. The isolated single-stranded RNA or DNA aptamer according to claim 1, for use in the diagnosis or prevention or therapeutic treatment of a TDP-43 proteinopathy.

14. The isolated single-stranded RNA or DNA aptamer for use according to claim 13, wherein the TDP-43 proteinopathy is selected from the group consisting of amyotrophic lateral sclerosis (ALS), frontotemporal lobar degeneration (FTLD), Alzheimer's disease, Lewy body dementia, Huntington's disease, argyrophilic grain dementia, Perry syndrome, progressive supranuclear palsy, corticobasal degeneration, and Pick's disease.

15. A pharmaceutical composition comprising a single-stranded RNA or DNA aptamer according to claim 1 and a pharmaceutically acceptable excipient.

16. A method of detecting in vitro the presence, absence or amount of TDP-43 aggregates in a sample, said method comprising detecting with a single-stranded RNA or DNA aptamer according to claim 1.

17. The method of claim 16, wherein the sample is selected from the group consisting of a TDP-43 preparation, a biological fluid or semi-fluid sample, a stool sample, a human or animal cell sample, and a human or animal tissue sample.

18. The method of claim 16, wherein the presence, absence or amount of TDP-43 aggregates is detected by optical microscopy detection, electronic microscopy detection, electro-optic detection, electrochemical detection, mass spectroscopy analysis, biochemical detection such as blotting.

19. A method of detecting in vitro the size of the TDP-43 aggregates in a sample from a subject affected by a TDP-43 proteinopathy, said method comprising detecting with a single-stranded RNA or DNA aptamer according to claim 1, wherein a size of the TDP-43 aggregates of from 10 to 100 nanometers is indicative of early stage TDP-43 proteinopathy, and wherein a size of the TDP-43 aggregates of from 0.250 to 1.50 micrometers is indicative of middle/late stage TDP-43 proteinopathy.

20. The method according to claim 19, wherein the sample is selected from the group consisting of post-mortem nervous system tissue, cerebrospinal fluid (CSF), blood, serum, plasma, lymphocytes and fibroblasts.

21. The method according to claim 19, wherein the size of the TDP-43 aggregates is determined by microscopy, light scattering or size exclusion chromatography.
